(19) United States
(12) Patent Application Publication
(10) Pub. No.: US 2015/0327818 A1
(43) Pub. Date: Nov. 19, 2015

(54) Positioning Device for Patients

(71) Applicant: Buck Engineering & Consulting GmbH, Reutlingen (DE)

(72) Inventors: Matthias Buck, Reutlingen (DE); Martin Gerlich, Stuttgart (DE); Mohamed Wassim Bouraoui, Stuttgart (DE)

(21) Appl. No.: 14/713,451

(22) Filed: May 15, 2015

(30) Foreign Application Priority Data
May 15, 2014 (EP) 14 001 711.2

(51) Int. Cl. A61B 6/04; A61G 13/04; A61G 13/06

(52) U.S. Cl. CPC A61B 6/0407; A61G 13/06; A61G 13/04

(57) ABSTRACT

A patient positioning device is provided with a patient support, a movement device, and a pivoting device connecting the patient support to the movement device. The movement device moves the patient support by enabling translational movements of the patient support in directions of the three spatial axes x, y, z of a coordinate system and rotational movements of the patient support about the three spatial axes x, y, z. The pivoting device enables a pivot movement of the patient support relative to the movement device about a horizontal pivot axis. The movement device is preferably a robot arm.

### BACKGROUND OF THE INVENTION

The invention relates to a positioning device for patients, comprising a movement device for movement of a patient support, wherein the movement device enables translational movement of the patient support in all three spatial directions and rotational movement about all three spatial axes.

US 2005/0234327 A1 discloses a patient positioning assembly which comprises a robot arm that has five rotational movement axes. The robot arm is supported on a track so as to be height-adjustable. The patient couch is arranged on the hand section of the robot arm which enables pivot movements of the patient support about all three spatial axes.

The invention has the object to provide a patient positioning device of the aforementioned kind that enables a variety of applications.

### SUMMARY OF THE INVENTION

In accordance with the present invention, this is achieved in that the patient support is connected to the movement device by means of a pivoting device which enables a pivot movement of the patient support relative to the movement device about a horizontal pivot axis.

It is thus provided that a pivoting device connects the patient support and the movement device and enables a pivot movement of the patient support relative to the movement device about a horizontal pivot axis. The additional horizontal pivot axis of the pivoting device enables pivoting of the movement device relative to the patient support, wherein the orientation of the patient support, for example, a horizontal orientation, is maintained. Accordingly, the relative position of the connection of the patient support to the movement device can be changed so that multiple uses are enabled.

The pivot axis of the pivoting device extends advantageously parallel to a longitudinal center axis of the patient support. The longitudinal center axis is the axis extending in the longitudinal direction of the patient support, in case of a patient couch, for example, in the longitudinal direction of the patient couch. Accordingly, since the pivot axis extends parallel to the longitudinal center axis, only minimal space is required for pivoting because the patient support pivots about its short side and not about its long side. In order to enable lateral pivoting of the movement device, it is provided that the pivoting device enables a pivot movement of the patient support relative to the movement device about an angle of at least 60° about the pivot axis of the pivoting device. Preferably, the pivoting device enables a pivot movement about at least 90° so that the movement device can be pivoted completely to the side. Particularly advantageous is a pivot angle of approximately 180°.

Advantageously, a low patient access height of the patient support can be realized by means of the pivoting device so that a patient can easily access and position himself on the patient support. For this purpose, the movement device is advantageously pivoted away from the area below the patient support. This lateral pivot movement can be realized by means of the movement device. The pivoting device pivots advantageously the patient support in opposite direction so that the patient support remains in the desired orientation, preferably horizontal orientation. In this way, it is possible to realize a very low patient access position. For radiation treatment, the movement device can be advantageously arranged partially below the patient support so that accessibility of the patient for treatment is not limited or impaired.

Advantageously, the pivot axis of the pivoting device coincides with the longitudinal center axis of the patient support. The patient support has advantageously a support surface for a patient. The support surface is the surface that supports the weight of the patient. The support surface is preferably in approximation a horizontally oriented and flat surface. However, the support surface can also be non-planar in order to provide a better supporting action for the patient, for example, a surface adapted to the contour of the patient. In case of a non-planar support surface the support surface is horizontally arranged when the patient resting on the support surface is positioned horizontally. When the support surface is horizontally arranged, the bottom side of the patient support is the side which is facing the floor of a room where the patient positioning device is installed. In order to be able to laterally pivot the movement device away within a small space, it is advantageously provided that the spacing of the pivot axis of the pivoting device relative to the floor of the room corresponds to or matches at least the spacing of the bottom side of the patient support to the floor of the room. The pivot axis in case of a horizontally arranged support surface can have a horizontal spacing and/or a vertical spacing to the longitudinal center axis of the patient support. When a vertical spacing is provided, the pivot axis of the pivoting device advantageously is positioned above the longitudinal center axis, i.e., at a greater spacing to the floor than the longitudinal center axis of the patient support.

Advantageously, the movement device is a robot arm. The robot arm has preferably at least six rotational movement axes. The robot arm enables advantageously translational movements in all spatial directions of the spatial axes and rotational movements about all spatial axes. The robot arm has advantageously a hand section which enables movements of the patient support about all three spatial axes. The pivoting device is advantageously secured on the hand section of the robot arm. The pivoting device is therefore arranged between the hand section of the robot arm and the patient support. In this way, the robot arm can be laterally pivoted away from the patient support and the pivoting device can carry out a rotational movement in opposite direction in order to maintain the orientation, preferably approximately horizontal orientation, of the patient support.

It is provided that the patient positioning device comprises a radiation treatment position in which the hand section is positioned at a spacing relative to the floor that is smaller than the spacing of the bottom side of the patient support relative to the floor. The hand section is therefore at least partially arranged below the patient support. Preferably, the entire hand section is arranged below the support surface, in particular below the patient support. Accordingly, the hand section is not in the way in case of radiation treatment of the patient. At the same time, a reduced radiation exposure of the hand section is achieved. The patient positioning device comprises advantageously a patient access position in which the spacing of the hand section relative to the floor is greater than the spacing of the bottom side of the patient support relative to the floor. The relative position of the hand section to the patient support changes thus between the radiation treatment position and the patient access position. Preferably, the hand section in the patient access position is arranged laterally adjacent to the patient support or above the patient support. Accordingly, the patient support can be lowered far down to the floor of the room and a patient can climb easily onto the patient support.

The robot arm comprises advantageously a base frame on which a carousel is supported so as to be rotatable about a first, in particular vertically arranged, rotational movement axis. The robot arm comprises a linkage (rocker arm) which relative to the carousel is supported pivotably about a second, preferably horizontally arranged, rotational movement axis. Advantageously, the robot arm comprises an arm section that relative to the linkage is pivotably supported about a third, in particular horizontally arranged, rotational movement axis. The first, second, and third rotational movement axes can however also be positioned at a slant relative to the horizontal direction and to the vertical direction in space.

In order to provide a patient access height as low as possible of the patient support in the patient access position, it is advantageously provided that the arm section is secured on the linkage by an intermediate section, wherein the intermediate section is supported on the linkage so as to be pivotable about a third rotational movement axis. The rotational movement of the intermediate section about the third rotational movement axis is advantageously coupled to the movement of the linkage about the second rotational movement axis by means of a coupling device. However, a separate drive for the rotational movement of the intermediate section about the third rotational movement axis may be advantageous. The second rotational movement axis and the third rotational movement axis are in particular oriented parallel relative to each other.

The configuration of the robot arm with an intermediate section between arm section and linkage constitutes an independent inventive concept that is independent of the pivoting device that enables a pivot movement of the patient support relative to the movement device about a horizontal pivot axis. On this arm section, advantageously a hand section is arranged that enables movements of the patient support about all three spatial axes.

The arm section is pivotably supported relative to the linkage in particular about a fourth, vertically arranged, rotational movement axis. Accordingly, the orientation of the arm section upon horizontal translational movement in a plane remains intact. Advantageously, the arm section is substantially horizontally oriented. Accordingly, the arm section can be moved in a horizontal plane at a minimal spacing relative to the floor of the room and the position of the patient access position can be flexibly selected. Since the rotational movement of the intermediate section about the third rotational movement axis is coupled to the movement of the linkage about the second rotational movement axis, an additional drive for the movement of the intermediate section can be omitted.

The coupling device can be a mechanical coupling device in this context. Preferably, the coupling device comprises a coupling element that is secured on the carousel at a spacing to the second rotational movement axis so as to be pivotable about a first bearing axis and is secured on the intermediate element at a spacing to the third rotational movement axis so as to be pivotable about a second bearing axis. Upon pivoting of the linkage relative to the carousel, the position of the first bearing axis relative to the linkage changes. Accordingly, the intermediate section is pivoted by means of the coupling element about the third rotational movement axis. The spacing of the first and second bearing axes relative to the associated rotational movement axes is advantageously the same so that the pivot angle of the intermediate section corresponds to the pivot angle of the linkage; in this way, the orientation of the arm section does not change.

In order to provide a patient access position as low as possible, the arm section is advantageously designed to be angled. The arm section has thus a first section by means of which the arm section is secured on the intermediate section and a second section on which the hand section is arranged. The spacing of the topside of the first section to the floor of the room is greater in this context than the spacing of the topside of the second section to the floor of the room. The area of the arm section which is facing the patient support is therefore displaced in a direction toward the floor relative to the area which is facing the intermediate section or the linkage. The displacement is preferably selected in this context to be so great that the patient support in the radiation treatment position can be rotated into a position above the arm section. Accordingly, in a simple way a comparatively free positioning of the patient support in space can be achieved.

### BRIEF DESCRIPTION OF THE DRAWING

FIG. 1 is a perspective illustration of a first embodiment of a patient positioning device in a radiation treatment position.

FIG. 2 is a side view of the patient positioning device of FIG. 1.

FIG. 3 is a perspective illustration of the patient positioning device of FIG. 1 upon pivoting into the patient access position.

FIG. 4 is a side view of the patient positioning device in the position illustrated in FIG. 3.

FIG. 5 shows in a perspective illustration the patient positioning device upon further pivoting.

FIG. 6 shows the patient positioning device in a side view in the position illustrated in FIG. 5.

FIG. 7 is a perspective illustration of the patient positioning device.

FIG. 8 is a side view of the patient positioning device of FIG. 7.

FIG. 9 is a perspective illustration of the patient positioning device in the patient access position.

FIG. 10 is a side view of the patient positioning device in the patient access position.

FIG. 11 shows a perspective illustration of the coupling device in a first position.

FIG. 12 shows a perspective illustration of the coupling device of FIG. 11 in a second position.

FIG. 13 is a perspective illustration of an embodiment of the patient positioning device in a radiation treatment position.

FIG. 14 shows the patient positioning device of FIG. 13 in a side view.

FIG. 15 is a perspective illustration of the patient positioning device of FIG. 13 upon pivoting into the patient access position.

FIG. 16 shows the patient positioning device of FIG. 15 in a side view.

FIG. 17 shows a side view of the patient positioning device of FIGS. 13 to 16 in the patient access position.

FIG. 18 is a perspective illustration of a further embodiment of the patient positioning device.

FIG. 19 shows the patient positioning device of FIG. 18 in a side view.

FIG. 20 shows the patient positioning device of FIG. 18 in the patient access position in perspective illustration.

FIG. 21 shows the patient positioning device in the position of FIG. 20 in a side view.

FIG. 22 shows a perspective illustration of an embodiment of the patient positioning device in a radiation treatment position.

FIG. 23 shows the patient positioning device of FIG. 21 in the patient access position in a perspective illustration.

FIG. 24 shows in a perspective illustration the patient positioning device in a further radiation treatment position.

FIG. 25 shows a perspective detail illustration of the patient positioning device of FIGS. 22 to 24.

### DESCRIPTION OF PREFERRED EMBODIMENTS

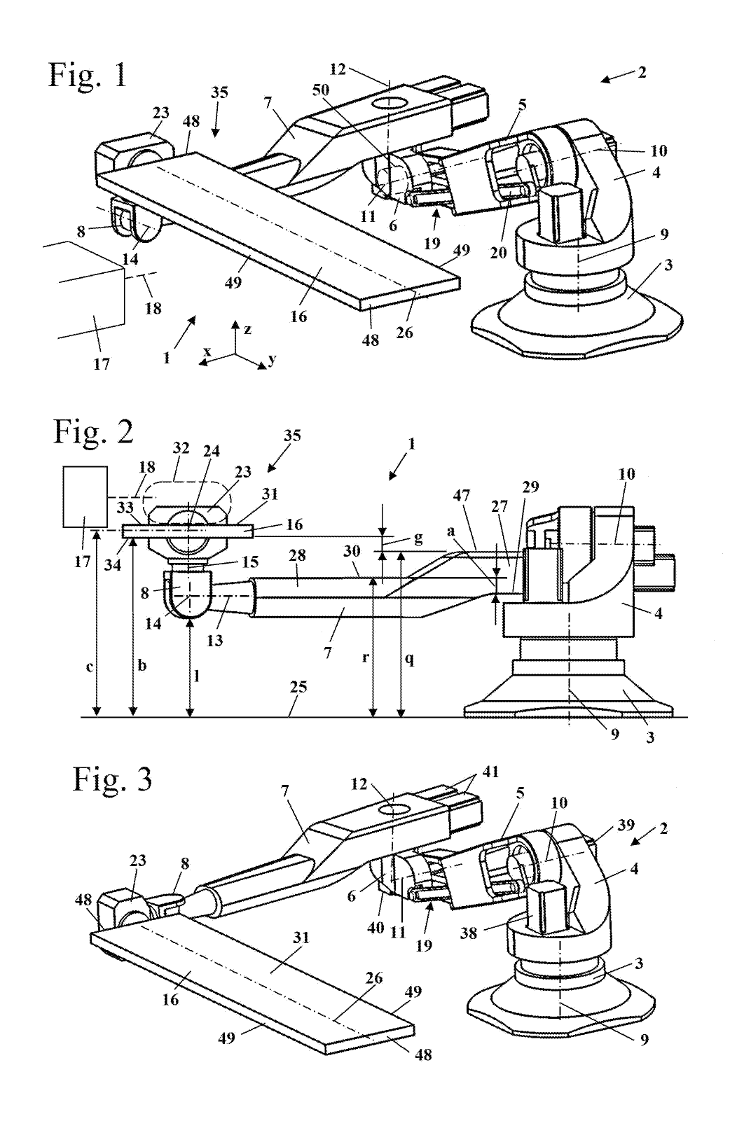

FIG. 1 shows a patient positioning device 1 and indicates also a spatial coordinate system x, y, z. The patient positioning device 1 serves for positioning a patient in a radiation device 17 which is schematically indicated in FIG. 1 and generates a schematically illustrated radiation beam 18 for radiation treatment of the patient. The radiation device 17 can comprise, for example, a radiation source for protons, neutrons or ions. The radiation device 17 can also be designed to generate a photon beam, for example, an x-ray or gamma radiation beam. Another type of radiation may also be advantageous. The patient positioning device 1 can additionally or alternatively also be used for positioning a patient in an imaging system, for example, an x-ray system, MRI or the like.

The patient positioning device 1 comprises a patient couch 16 which is secured on a movement device. In the embodiment, the movement device is a robot arm 2. A different type of movement device, for example, a movement device with several translational movement axes, may be advantageous. Instead of the patient couch 16, a different type of patient support such as a chair or the like may be provided also. The robot arm 2 comprises a base frame 3 on which a carousel 4 is supported so as to be rotatable about a first, vertically oriented, rotational movement axis 9. On the carousel 4, a linkage 5 is pivotably supported about a second axis of rotation (second rotational movement axis) 10. The second rotational movement axis 10 is horizontally arranged. The robot arm 2 comprises an arm section 7 on which a hand section 8 is arranged. Between the linkage 5 and the arm section 7, an intermediate section 6 is arranged. The intermediate section 6 is pivotably supported relative to the linkage 5 about a third, horizontally arranged, rotational movement axis 11. The rotational movement axes 10 and 11 extend parallel to each other.

The first rotational movement axis 9 can also be slanted relative to the vertical direction. The second rotational movement axis 10 and the third rotational movement axis 11 can also be slanted relative to the horizontal direction. The first rotational movement axis 9 is positioned relative to the second and third rotational movement axes 10, 11 at an angle of more than 0°, in particular 90°, in order to enable movements in all three spatial directions.

In the embodiment, the intermediate section 6 is pivoted about the same pivot angle as the linkage 5 but in opposite direction relative to the linkage 5. For transmitting the pivot movement, a coupling device 19 is provided which comprises a coupling rod 20. When the linkage 5 is pivoted upwardly, the intermediate section 6 is pivoted downwardly by the same pivot angle in opposite direction so that the orientation of the intermediate section 6 is maintained. However, a separate drive 50 for pivoting the intermediate section 6 on the linkage 5 may be provided as is schematically indicated in FIG. 1. On the intermediate section 6, the arm section 7 is pivotably supported about a fourth rotational movement axis 12. The fourth rotational movement axis 12 is vertically oriented. Due to coupling of the pivot movement of linkage 5 and of intermediate section 6, the vertical orientation of the fourth rotational movement axis 12 is maintained upon pivot movements of the linkage 5 about the second rotational movement axis 10.

The patient couch 16 is supported on the hand section 8 by means of a pivoting device 23. As can be seen in FIG. 1, the patient couch 16 has a substantially rectangular shape. The patient couch 16 has two opposite short sides 48 and two opposite long sides 49, as can be seen in FIG. 3. The long sides 49 are longer than the short sides 48. The pivoting device 23 is secured on one of the short sides 48 of the patient couch 16. The patient couch 16 has a longitudinal center axis 26 which extends parallel to the long sides 49 and centrally between the long sides 49. In FIG. 1, a rotational movement axis 14 of the hand section 8 is indicated also.

FIG. 2 shows the configuration of the hand section 8 in detail. The hand section 8 is rotatably supported relative to arm section 7 about a fifth rotational movement axis 13 of the robot arm 2. The fifth rotational movement axis 13 extends in longitudinal direction of the arm section 7 and, due to the horizontal orientation of the arm section 7, is also horizontally oriented. The hand section 8 comprises a sixth rotational movement axis 14 which is oriented perpendicular to the fifth rotational movement axis 13 as well as a seventh rotational movement axis 15 which is perpendicular to the sixth rotational movement axis 14. In the arrangement illustrated in FIG. 2, the fifth rotational movement axis 13 and the seventh rotational movement axis 15 are oriented approximately perpendicular to each other. The hand section 8 projects approximately perpendicularly upwardly.

As a result of the rotational movement axes 9 to 15, the robot arm 2 enables movements of the patient couch 16 in all three spatial directions, i.e., translational movements in the direction of the spatial axes x, y, z and also rotational movements about all three spatial axis x, y, z of the spatial coordinate system.

A patient 32 is illustrated schematically in FIG. 2 resting on the patient couch 16. The patient 32 is resting in this context on a support surface 31 of the patient couch 16 which is formed by the topside 33 of the patient couch 16. In FIG. 2, the patient couch 16 is arranged with its support surface 31 extending horizontally. The support surface 31 in the embodiment is flat. A curved or irregularly shaped support surface 31 may be advantageous also. FIGS. 1 and 2 show the patient positioning device 1 in a radiation treatment position 35. The patient couch 16 comprises a bottom side 34 which is facing the floor 25 of the room where the patient positioning device 1 is installed and is spaced in the radiation treatment position 35 relative to the floor 25 at a spacing b. In the radiation treatment position 35, the hand section 8 is arranged below the plane of the support surface 31 and also below the plane of the bottom side 34 of the patient couch 16. The hand section 8 is spaced at a spacing l relative to the floor 25 which is significantly smaller than the spacing b of the bottom side 34 of the patient couch 16 relative to the floor 25. The pivoting device 23 enables pivoting of the patient couch 16 relative to the hand section 8 about a pivot axis 24. In the patient positioning device 1 the pivot axis 24 coincides with the longitudinal center axis 26 of the patient couch 16. The pivot axis 24 has in the illustrated radiation treatment position 35 shown in FIG. 2 in which the support surface 31 is oriented horizontally to the floor 25 a spacing c that is somewhat greater than the spacing b of the bottom side 34 of the patient couch 16 to the floor 25 of the room.

In FIG. 2, the configuration of the arm section 7 is also shown in detail. The arm section 7 is angled and comprises a first section 27 that is arranged on the intermediate section 6 (FIG. 1) as well as a second section 28 on which the hand section 8 is secured. The second section 28 is displaced relative to the first section 27 in the direction toward the floor 25 of the room. The first section 27 has a topside 47 which is facing away from the floor 25 and is spaced at a spacing q relative to the floor 25. The second section 28 has a topside 30 whose spacing r to the floor 25 is smaller than the spacing q. These sections 27 and 28 are oriented each approximately horizontally relative to the floor 25 of the room. As shown in FIG. 2, the first and second sections 27, 28 of the angled arm section 7 are displaced by less than the height measurement of the first section 27 measured vertically. The topside 30 of the second section 28 is positioned above a bottom side 29 of the first section 27. As shown in FIG. 2, the topside 30 has a spacing a relative to the bottom side 29. The arm section 7 is designed such that the patient couch 16 in the radiation treatment position 35 illustrated in FIGS. 1 and 2 can pivot to a position above the arm section 7. The topside 47 of the first section 27 is for this purpose arranged at a smaller spacing relative to the floor 25 than the bottom side 34 of the patient couch 16. The spacing q is smaller than the spacing b. Between the bottom side 34 and the topside 47 of the arm section 7, a spacing g is formed which is vertically measured and corresponds to the difference between the spacing b and the spacing q. The spacing g is advantageously sized such that the patient couch 16 even for a deformation of the patient couch 16, for example, due to the weight of the patient 32, is still capable of being pivoted into a position above the arm section 7.

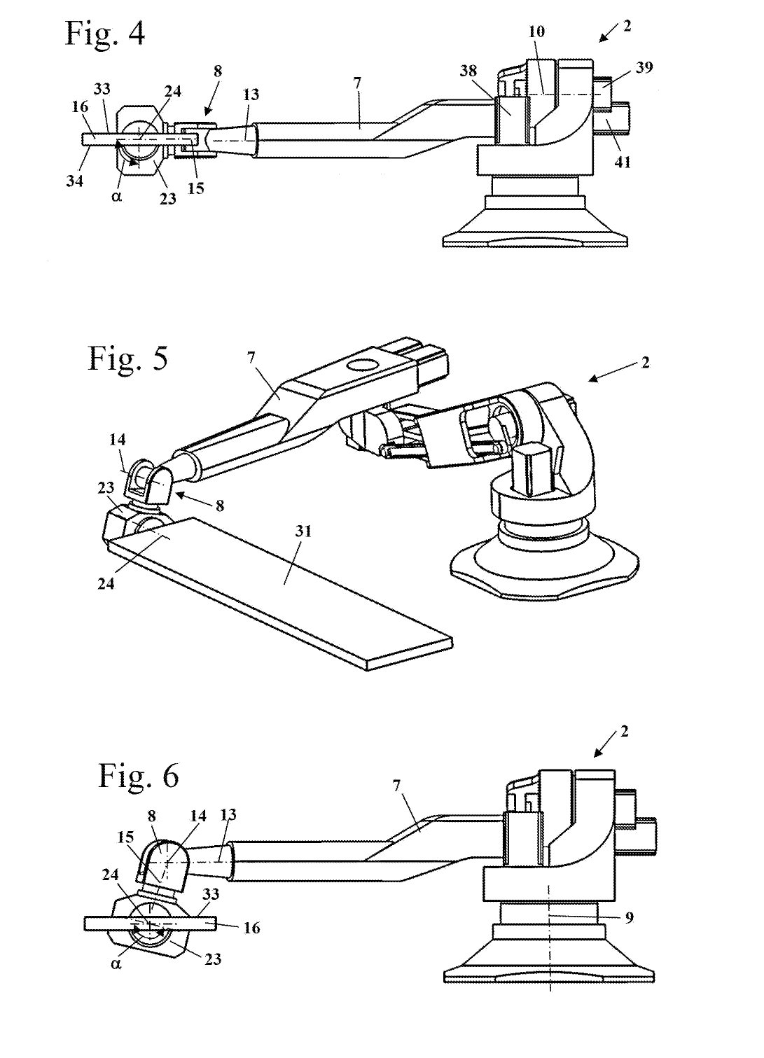

FIGS. 3 and 4 show the patient positioning device 1 after pivoting of the pivoting device 23 relative to the patient couch 16 about a pivot angle α of approximately 90°. In order for the horizontal orientation of the support surface 31 to be maintained, the hand section 8 has carried out an appropriate pivot movement in opposite direction wherein in the embodiment pivoting of the hand section 8 has been realized about all three rotational movement axes 13, 14, 15. The rotational movement axes 13 and 15 of the hand section 8 are positioned parallel to each other in the position shown in FIGS. 3 and 4. In FIG. 3 the drives of the robot arm 2 are also shown. The robot arm 2 comprises a first drive 38 for pivoting the carousel 4 relative to the base frame 3. The drive 39 is provided for pivoting the linkage 5 relative to the carousel 4. In FIG. 3, a third drive 40 is also partially visible that is serving for pivoting the arm section 7 relative to the intermediate section 6. On the arm section 7, two drives 41 are arranged which pivot the hand section 8. As can be seen also in FIG. 3, no additional drive is provided for pivoting the intermediate section 6 relative to the linkage 5. The pivoting action of the intermediate section 6 about the rotational movement axis 11 is realized by means of the coupling device 19.

FIGS. 5 and 6 show the robot arm 2 after further pivoting of the pivoting device 23 and of the hand section 8. Compared to the radiation treatment position 35 of FIG. 2, the pivoting device 23 has pivoted the patient couch 16 about a pivot angle α of somewhat less than 180°. The rotational movement axis 15 of the hand section 8 is positioned at a slant. The hand section 8 has carried out a pivot movement in opposite rotational direction so that the horizontal orientation of the patient couch 16 has been maintained. In the arrangement illustrated in FIGS. 5 and 6, the hand section 8 is already completely arranged above the support surface 31 or the topside 33 of the patient couch 16.

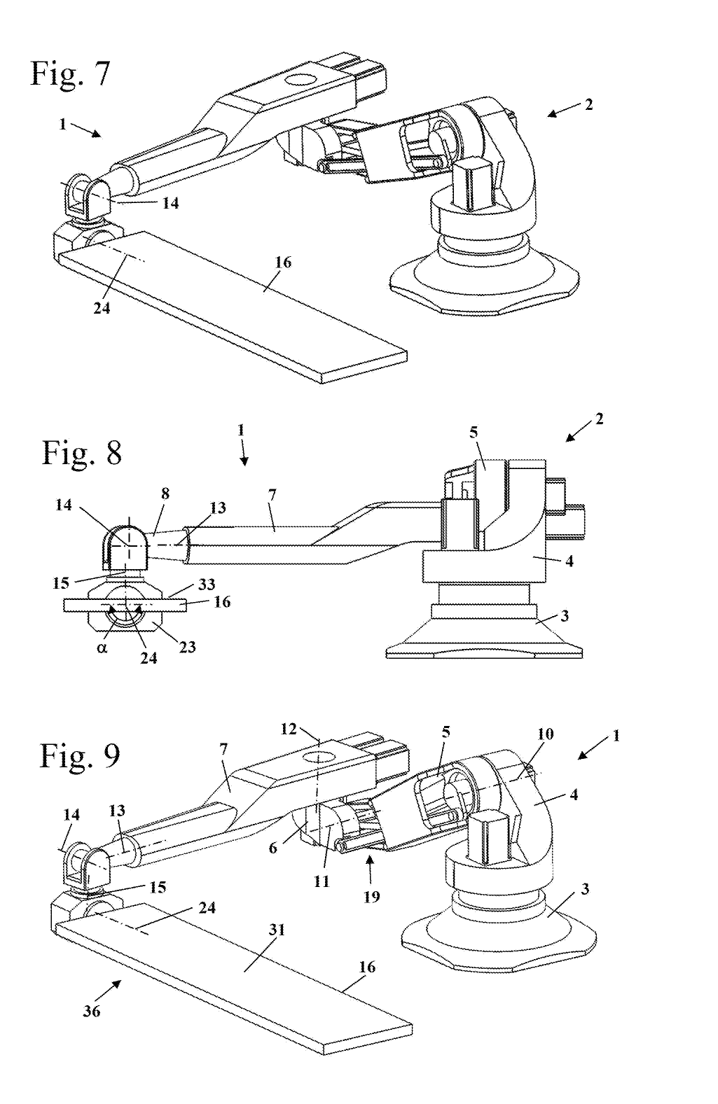

FIGS. 7 and 8 show the patient positioning device 1 after further pivoting of the patient couch 16 about the pivot axis 24 of the pivoting device 23. In the position illustrated in FIGS. 7 and 8, the pivoting device 23 has carried out a pivoting action about the pivot angle α of 180° relative to the radiation position 35. The rotational movement axis 15 of the hand section 8 is arranged perpendicular to the rotational movement axis 13 and is vertically oriented in space. In FIGS. 1 to 8 the position of the arm section 7 is unchanged.

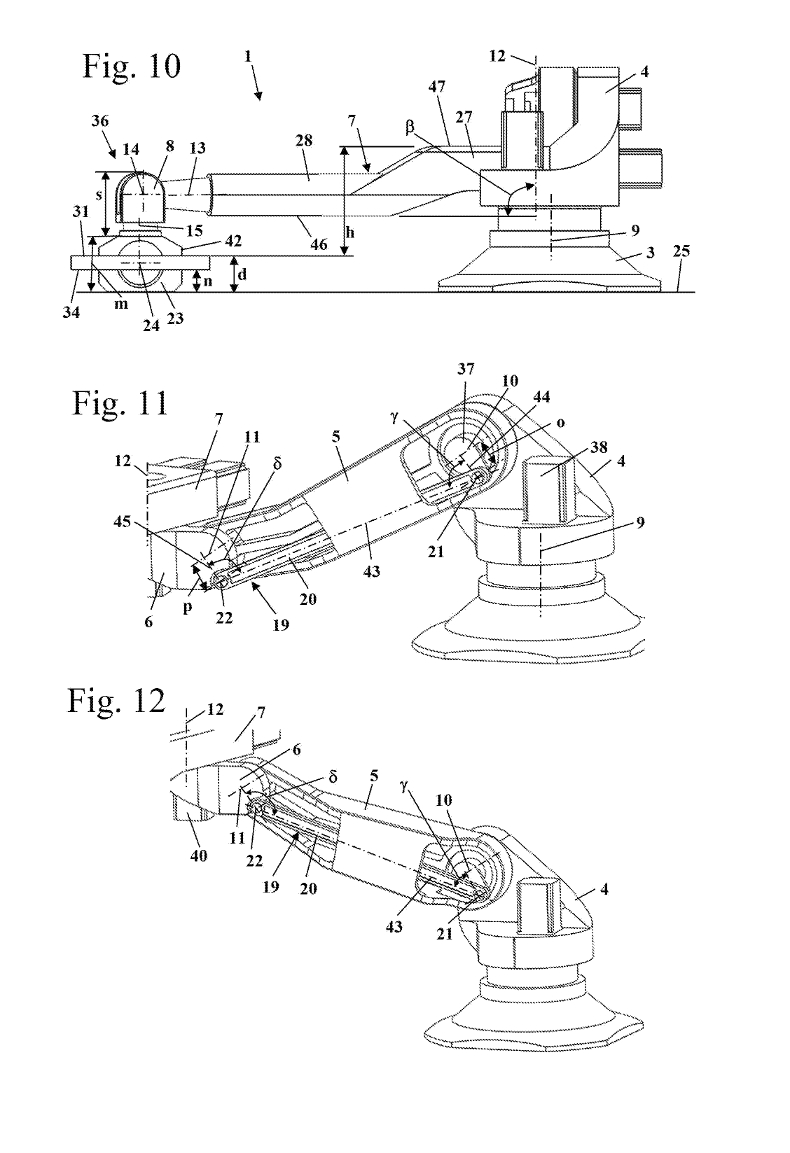

FIGS. 9 and 10 show the patient positioning device 1 in a patient access position 36. In this position, the support surface 31 has a minimal spacing d relative to the floor 25 of the room. The pivoting device 23 has been lowered into a position that is close to the floor 25 of the room. The pivoting device 23 has a housing 42 which is arranged on the floor 25 in the patient access position 36. The hand section 8 has a spacing m relative to the floor 25 that is significantly greater than a spacing n of the bottom side 34 of the patient couch 16 relative to the floor 25 and also significantly greater than the spacing d (FIG. 10). The support surface 31 is arranged in the patient access position 36 below the topside 47 of the arm section 7 and has relative thereto a vertical spacing h. As shown in FIG. 10, the bottom side 46 of the second section 28 of the arm section 7 is parallel to the floor 25, i.e., horizontal, and perpendicular to the fourth rotational movement axis 12 of the arm section 7. Accordingly, the arm section 7 can be pivoted in the radiation treatment position 35 (FIG. 2) as well as in the patient access position 36 (FIG. 10) about the rotational movement axis 12 and 9; in this way, it can be moved in a horizontal plane in space. Since the bottom side 46 is oriented parallel to the floor 25 of the room, the spacing to the floor 25 can therefore be very small. In the patient access position 36, the bottom side 46 of the second section 28 of the arm section 7 is positioned at an angle β relative to the rotational movement axis 12 that is approximately 90°.

As can be seen in FIG. 10, the spacing d of the support surface 31 relative to the floor 25 in the patient access position 36 is significantly smaller than the height s of the hand section 8 that is measured in vertical direction for vertically oriented rotational movement axis 15. Since the hand section 8 is arranged above the patient couch 16, the support surface 31 can be arranged at a spacing to the floor 25 which is smaller than the height s of the hand section 8.

FIGS. 11 and 12 show the configuration of the coupling device 19 in detail. The carousel 4 has a hub 37 where a first bearing axis 21 for the coupling rod 20 is provided. The first bearing axis 21 has relative to the rotational movement axis 10 of the linkage 5 a spacing o. In the position illustrated in FIG. 11 a connection 44 between the rotational movement axis X and the first bearing axis 21 is positioned at an angle γ relative to a longitudinal center axis 43 of the coupling rod 20; this angle γ in the embodiment is somewhat smaller than 90°. The coupling rod 20 is supported with its second end on a second bearing axis 22 at the intermediate section 6 so as to be pivotable. The second bearing axis 22 is positioned at a spacing p relative to the rotational movement axis 11 of the intermediate section 6. The spacings o and p are preferably of the same size. The rotational movement axis 11 is connected with the second bearing axis 22 by means of a connection 45 which is positioned relative to the longitudinal center axis 43 of the coupling rod 20 at an angle δ. The connections 44 and 45 define a parallelogram together with the longitudinal center axis 43 and a connecting line of the rotational movement axes 10 and 11. The sum of the angles γ and δ is 180° in every position. Accordingly, the vertical orientation of the rotational movement axis 12 upon pivoting of the linkage 5 about the second rotational movement axis 10 is maintained without an additional drive being required for the intermediate section 6. The imaginary connections 44 and 45 are indicated in a plane perpendicular to the bearing axis 21 and 22.

In the position illustrated in FIG. 12, the intermediate section 6 has moved upwardly while the linkage 5 has been pivoted about rotational movement axis 10 in FIGS. 11 and 12 in clockwise direction. The angle γ is reduced and the angle δis enlarged appropriately. The rotational movement axis 12 of the arm section 7 is still vertically arranged. The position of the first bearing axis 21 relative to the carousel 3 is unchanged. The linkage 5 has moved relative to the first bearing axis 21.

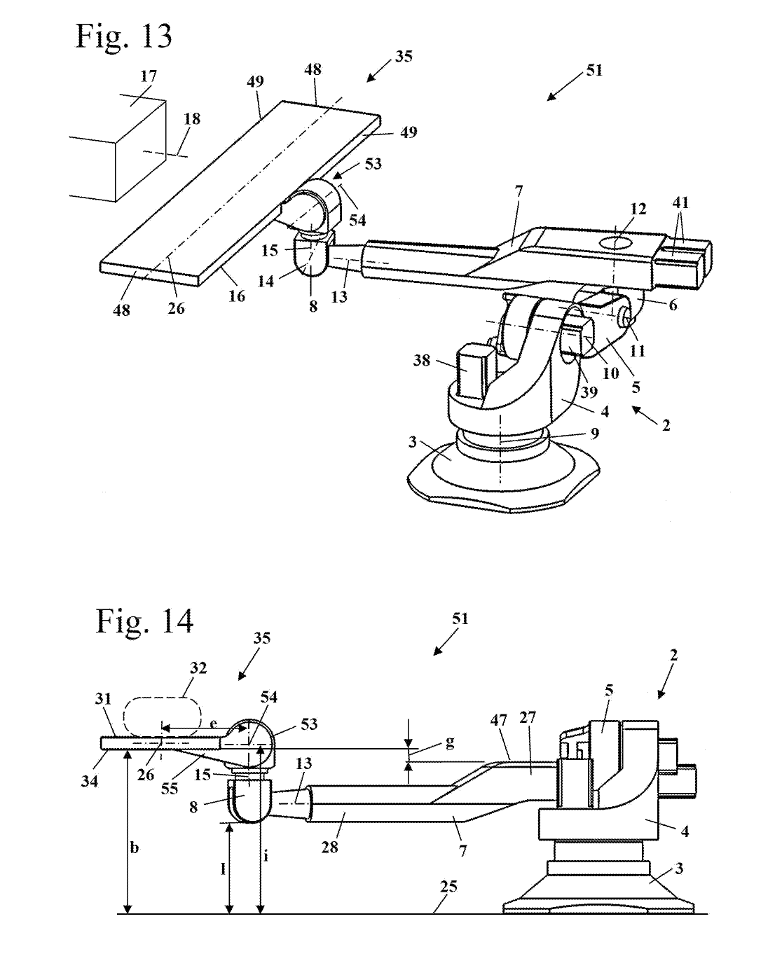

FIGS. 13 to 17 show an embodiment of a patient positioning device 51. Same reference characters indicate in all Figures components that correspond to each other wherein reference is being had to the description of those components in connection with FIGS. 1 to 12. The robot arm 2 that serves for positioning the patient couch 16 of the patient positioning device 51 corresponds to the robot arm 2 of the embodiment of FIGS. 1 to 12.

FIGS. 13 and 14 show the patient positioning device 51 in a radiation treatment position 35. The patient couch 16 is secured by means of a pivoting device 53 on the hand section 8 of the robot arm 2. The pivoting device 53 comprises a pivot axis 54 which extends parallel to the longitudinal center axis 26 of the patient couch 16. As can be seen in FIG. 14, the pivot axis 54 has relative to the longitudinal center axis 26 of the patient couch 16 a horizontal spacing e that is greater than half the width of the patient couch 16. The pivot axis 54 is therefore arranged adjacent to the patient couch 16. As is shown also in FIG. 14, the pivoting device 53 comprises a support arm 55 which extends on the bottom side 34 of the patient couch 16 and serves for stiffening and better fixation of the patient couch 16 on the pivoting device 53.

As shown in FIG. 14 the spacing b of the bottom side 34 of the patient couch 16 in the radiation treatment position 35 is significantly greater than spacing l of the hand section 8 relative to the floor 25 of the room. The hand section 8 is positioned underneath the plane of the bottom side 34 of the patient couch 16. The pivot axis 54 comprises relative to the floor 25 a spacing i which in the embodiment is somewhat greater than the spacing b of the bottom side 34 relative to the floor 25 of the room. The bottom side 34 extends in a plane above the topside 47 of the arm section 7. Between the topside 47 of the arm section 7 and the bottom side 34 of the patient couch 16 there is a vertical spacing g so that the patient couch 16, even in case of being bent downwardly, for example, due to the weight of the patient, is still pivotable into a position above the arm section 7.

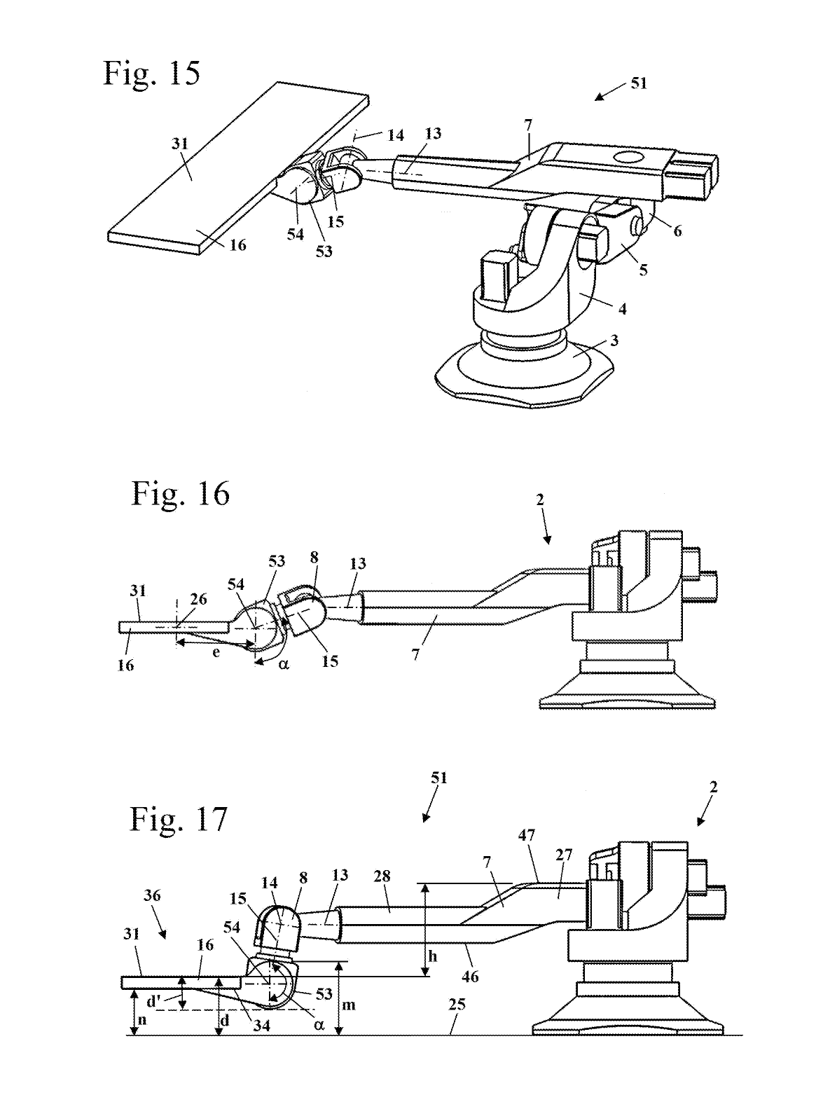

FIGS. 15 and 16 show the patient couch 16 as it is pivoted into a patient access position 36 (FIG. 17). In the radiation treatment position 35 illustrated in FIGS. 13 and 14, the rotational movement axis 15 of the hand section 8 extends perpendicular to the rotational movement axis 13 of the hand section 8. The hand section 8 in this context is upwardly oriented and the pivoting device 53 is arranged above the hand section 8. In FIGS. 15 and 16, the pivoting device 53 has pivoted the patient couch 16 about a pivot angle α that is greater than 90° about pivot axis 54. The hand section 8 has performed an appropriate pivot movement in opposite direction about a spatial axis that is extending parallel thereto so that the horizontal orientation of the support surface 31 has been maintained. Pivoting about the axis that is parallel to the pivot axis 54 can be realized by a combined rotational movement about the rotational movement axes 13, 14, and 15 of the hand section 8. It can also be provided that the rotational movement axis 14 is oriented parallel to the pivot axis 54 and, subsequently, an appropriate adjustment is achieved by pivoting the hand section 8 about the rotational movement axis 14 and pivoting of the patient couch 16 about the pivot axis 54 by the same angle in opposite direction.

FIG. 17 shows the patient positioning device 51 in the patient access position 36. In a patient access position 36, the support surface 31 has a spacing d to the floor 25 which is comparatively small. Relative to the radiation treatment position 35 illustrated in FIG. 14, the pivoting device 53 has performed a pivoting action about a pivot angle α that in the embodiment is somewhat smaller than 180°. However, it may be provided also that the pivoting device 53 carries out pivoting about a pivot angle α of 180°. In the embodiment, the pivoting device 53 has a spacing relative to the floor 25 in the patient access position 36. For reducing the access height, the arm section 7 with the hand section 8, the pivoting device 53, and the patient couch 16 can still be lowered by pivoting the linkage 5 about the rotational movement axis 10 and appropriate pivoting of the intermediate section 6 in opposite direction until the pivoting device 53 is arranged on the floor 25 in order to reduce the spacing d to a spacing d′ which is schematically illustrated in FIG. 17. As is also shown in FIG. 17, the hand section 8 has a spacing m to the floor 25 which is greater than the spacing d. The hand section 8 is positioned completely above the plane of the support surface 31. The support surface 31 is arranged below the topside 47 of the arm section 7 at a vertical spacing h relative to the topside 47. The support surface 31 in the embodiment is arranged at a smaller spacing to the floor 25 than the bottom side 46 of the second section 28 of the arm section 7.

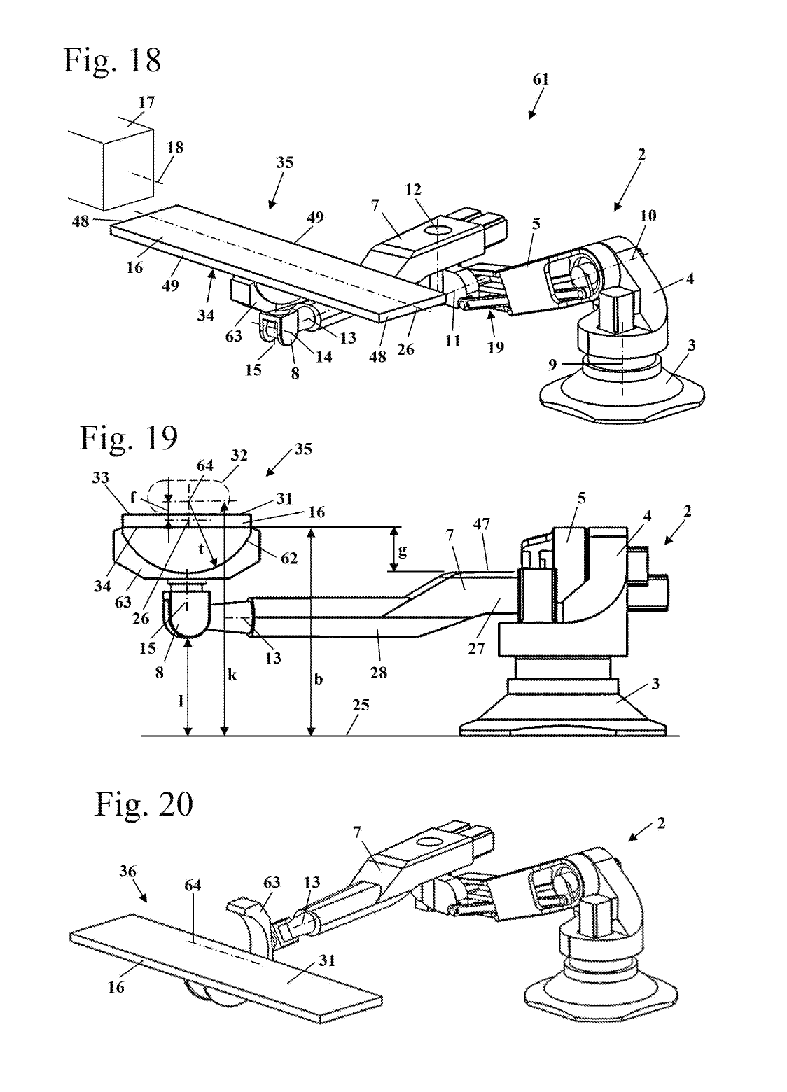

In the embodiment of the patient positioning device 61 illustrated in FIGS. 18 to 21, between the robot arm 2 and the patient couch 16 a pivoting device 63 is provided which is arranged at the bottom side 34 of the patient couch 16. The pivoting device 63 has a curved guide 62 (FIG. 19) which enables pivoting of the patient couch 16 about pivot axis 64. As is shown in FIG. 19, the pivot axis 64 in the radiation treatment position 35 has a spacing k to the floor 25 that is greater than the spacing b of the bottom side 34 of the patient couch 16 relative to the floor 25 of the room. The hand section 8 in the radiation treatment position 35 is arranged underneath the pivoting device 63. The rotational movement axis 13 of the hand section 8 is horizontally arranged and the rotational movement axis 15 is vertically arranged. The pivot axis 64 of the pivoting device 63 is positioned above the support surface 31. In the embodiment, the pivot axis 64 is located perpendicularly above the longitudinal center axis 26 of the patient couch 16 and is arranged at a vertical spacing f relative to the longitudinal center axis 26 and parallel to the longitudinal center axis 26. As is also shown in FIG. 19, a radius t of the guide 62 is greater than half the width of the patient couch 16. In this way, the pivoting device 63 can be arranged centrally at the patient couch 16, i.e., neighboring the center of the long sides 49, and the patient couch 16 can project through the pivoting device 63. The hand section 8 is arranged underneath the patient couch 16 in the radiation treatment position 35. The spacing l of the hand section 8 to the floor 25 is significantly smaller than the spacing b of the bottom side 34 of the patient couch 16 relative to the floor 25 of the room. Also, the pivoting device 63 is completely arranged underneath the patient couch 16. The bottom side 34 of the patient couch 16 is positioned in a plane which is arranged above the topside 47 of the arm section 7. In this way, the patient couch 16 has a vertical spacing g to the arm section 7 so that the patient couch 16 can be pivoted into a position above the arm section 7.

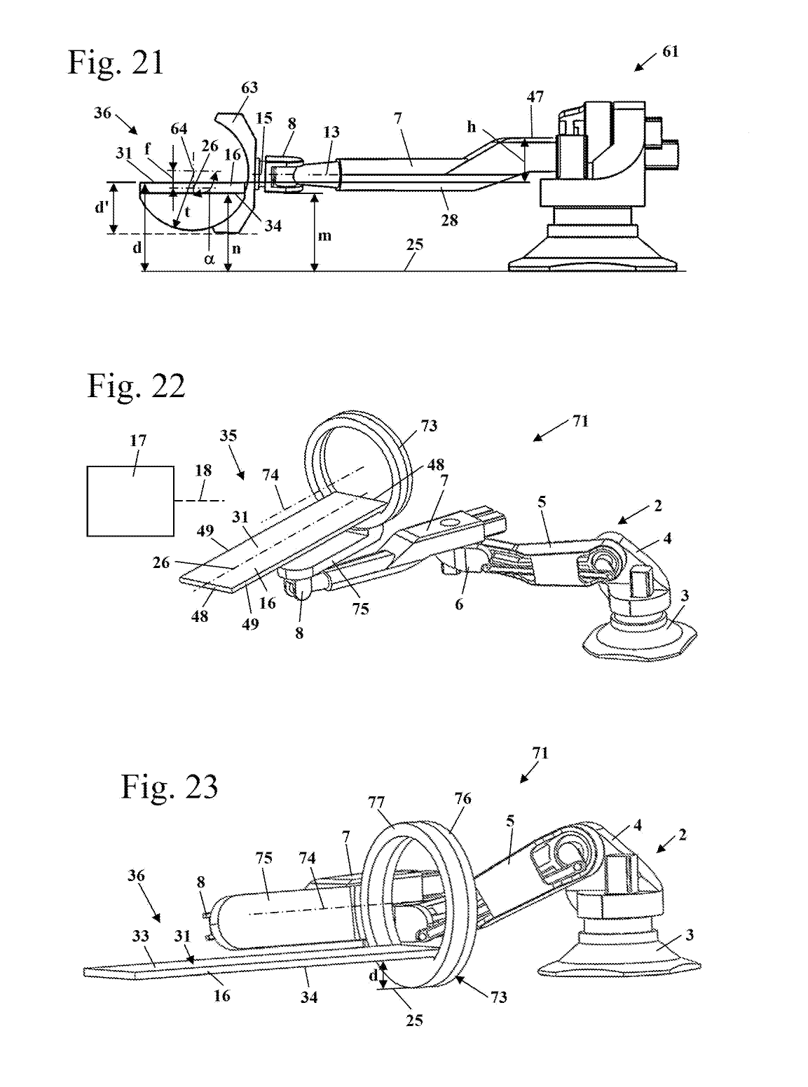

FIGS. 20 and 21 show the patient positioning device 61 in the patient access position 36. In this position, the pivoting device 63 has pivoted the patient couch 16 about pivot angle α that is approximately 90°. The hand section 8 has carried out a pivot movement in opposite direction so that the horizontal orientation of the support surface 31 of the patient couch 16 is maintained. The bottom side 34 of the patient couch 16 has a spacing n relative to the floor 25 that is somewhat smaller than the spacing m of the hand section 8 relative to the floor 25. The rotational movement axes 13 and 15 of the hand section 8 are parallel to each other and horizontally oriented. The hand section 8 is arranged in the area adjacent to the patient couch 16. The support surface 31 has relative to the floor 25 a spacing d. By pivoting the linkage 5, the patient couch 16 can be further lowered until the support surface 31 has a schematically indicated spacing d′ (see FIG. 21) relative to the floor 25 of the room. The spacing d′ is determined also by the pivoting device 63, i.e., the radius t of the pivoting device 63. In the patient access position 36, the support surface 31 is arranged underneath the topside 47 of the arm section 7 and has relative thereto a vertical spacing h. The patient couch 16 is arranged approximately at the level of the section 28 of the arm section 7.

FIGS. 22 to 25 show another embodiment of a patient positioning device 71. The patient positioning device 71 has a pivoting device 73 by means of which the patient couch 16 is secured on the hand section 8 of the robot arm 2. The pivoting device 73 is arranged adjacent to a short side 48 of the patient couch 16. The hand section 8 is arranged approximately centrally underneath the patient couch 16 and connected by a carrier 75 to the pivoting device 73. In this way, the bending moments introduced into the arm section 7 can be reduced. The pivoting device 73 enables pivoting of the patient couch 16 about pivot axis 74 which is positioned parallel to the longitudinal center axis 26 of the patient couch 16. FIG. 25 shows the arrangement in the radiation treatment position 35. In this position, the hand section 8 is arranged underneath the patient couch 16. Also, the carrier 75 is located underneath the patient couch 16.

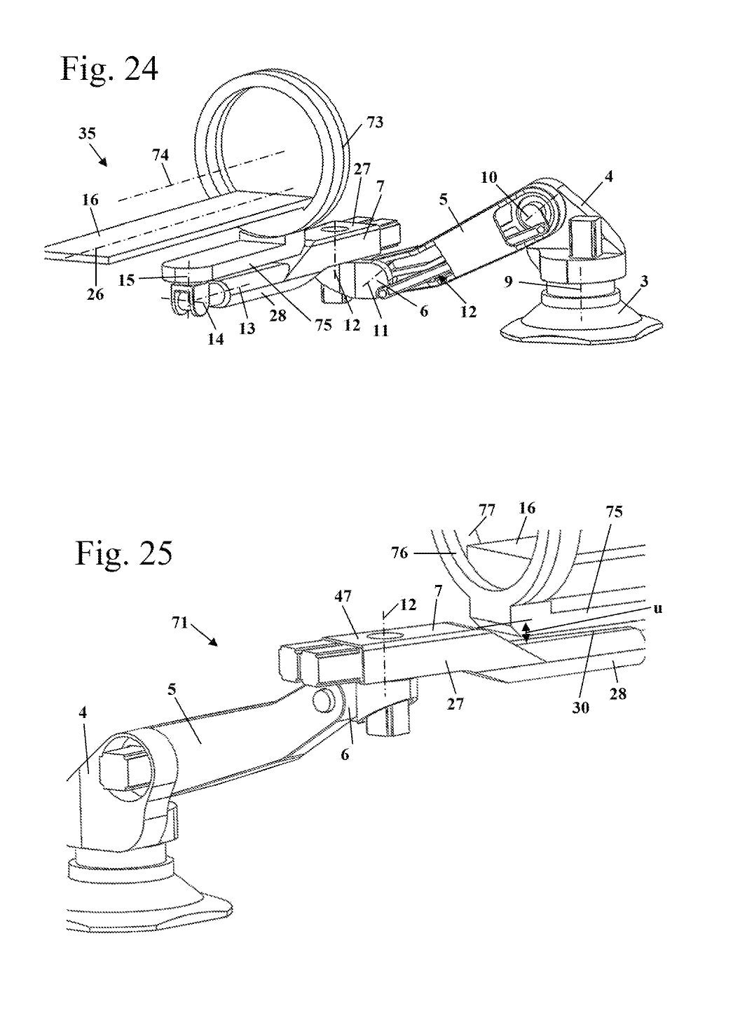

FIG. 23 shows the arrangement in the patient access position 36. The pivoting device 73 has pivoted the patient couch 16 about pivot axis 74 by about 90°. The robot arm 2 has pivoted the carrier 75 in opposite direction so that the horizontal orientation of the patient couch 16 has been maintained. The carrier 75, like arm section 7, is arranged in this access position 36 laterally adjacent to the patient couch 16 and above the plane of the topside 33 of the patient couch 16. The pivoting device 73 comprises a first ring 76 which is secured on the carrier 75 as well as a second ring 77 which is secured on the patient couch 16. The inner diameters of the rings 76 and 77 are greater than the width of the patient couch 16. The minimal access height g is predetermined by the vertical spacing of the bottom side 34 of the patient couch 16 relative to the bottom side of the rings 76 and 77, i.e., the side of the rings 76 and 77 facing the floor 25, for horizontally oriented support surface 31. In the access position 36, the hand section 8 is positioned above the plane of the support surface 31. As is shown in FIGS. 24 and 25, the arm section 7 is angled such that the carrier 75 can be pivoted into a position above the arm section 7. As is shown in FIG. 25, the topside 47 of the first section 27 of the arm section 7 has a vertical spacing u relative to the topside 30 of the second section 28 of the arm section 7. In this way, the patient couch 16 can be rotated freely about rotational movement axis 15 about 360°. At the same time, as a result of the angled configuration of the arm section 7, a low radiation treatment position 35 can be achieved.

It may be provided that the patient couch 16 is arranged on the pivoting device 73 so as to be horizontally movable by a translational movement. On the patient couch 16 additionally an imaging device can be arranged. The imaging device must not be mandatorily pivotable relative to the hand section 8 for reaching the patient access position 36 but can also be arranged fixedly on the hand section 8 and may even allow for pivoting about a pivot axis which is parallel to the longitudinal center axis 26 so that the elements of the imaging device can be pivoted away laterally for allowing patient access.

The specification incorporates by reference the entire disclosure of European priority document 14 001 711.2 having a filing date of May 15, 2014.

While specific embodiments of the invention have been shown and described in detail to illustrate the inventive principles, it will be understood that the invention may be embodied otherwise without departing from such principles.

What is claimed is:

1. A patient positioning device comprising:
a patient support;
a movement device;
a pivoting device connecting the patient support to the movement device;
wherein the movement device is configured to move the patient support by enabling translational movements of the patient support in directions of the three spatial axes (x, y, z) of a coordinate system and rotational movements of the patient support about the three spatial axes (x, y, z);
wherein the pivoting device is configured to enable a pivot movement of the patient support relative to the movement device about a horizontal pivot axis.

2. The patient positioning device according to claim 1, wherein the horizontal pivot axis of the pivoting device extends parallel to a longitudinal center axis of the patient support.

3. The patient positioning device according to claim 1, wherein the pivot movement of the patient support relative to the movement device about the horizontal pivot axis of the pivoting device comprises a pivot angle of at least 60°.

4. The patient positioning device according to claim 1, wherein the horizontal pivot axis of the pivoting device coincides with a longitudinal center axis of the patient support.

5. The patient positioning device according to claim 1, wherein the patient support comprises of support surface for a patient and further comprises a bottom side, wherein the bottom side, when the support surface is horizontally arranged, is facing a floor, wherein a vertical spacing of the horizontal pivot axis of the pivoting device relative to the floor corresponds at least to a spacing of the bottom side relative to the floor.

6. The patient positioning device according to claim 5, wherein the horizontal pivot axis of the pivoting device has a horizontal spacing relative to a longitudinal center axis of the patient support when the support surface is horizontally arranged.

7. The patient positioning device according to claim 5, wherein the horizontal pivot axis of the pivoting device has a vertical spacing relative to a longitudinal center axis of the patient support when the support surface is horizontally arranged.

8. The patient positioning device according to claim 1, wherein the movement device is a robot arm.

9. The patient positioning device according to claim 8, wherein the robot arm comprises at least six rotational movement axes, wherein the robot arm comprises a hand section configured to enable movements of the patient support about said three spatial axes, and wherein the pivoting device is secured on the hand section of the robot arm.

10. The patient positioning device according to claim 9, wherein the patient support comprises of support surface for a patient and further comprises a bottom side, wherein the bottom side, when the support surface is horizontally arranged, is facing a floor, wherein the patient positioning device comprises a radiation treatment position and a patient access position, wherein in the radiation treatment position the hand section has a vertical spacing relative to the floor and the vertical spacing of the hand section relative to the floor is smaller than a spacing of the bottom side relative to the floor, and wherein in the patient access position the vertical spacing of the hand section relative to the floor is greater than a spacing of the bottom side of the patient support relative to the floor.

11. The patient positioning device according to claim 8, wherein the robot arm comprises a base frame and a carousel supported on the base frame so as to be pivotable about a first rotational movement axis and wherein the robot arm has a linkage that is pivotably supported about a second rotational movement axis relative to the carousel.

12. The patient positioning device according to claim 11, wherein the robot arm comprises an arm section that is pivotably supported about a third rotational movement axis relative to the linkage, wherein the third rotational movement axis is horizontally arranged.

13. A patient positioning device comprising:
a robot arm configured to move a patient support by enabling translational movements of the patient support in directions of the three spatial axes (x, y, z) of a coordinate system and rotational movements of the patient support about the three spatial axes (x, y, z);
wherein the robot arm comprises a base frame and a carousel pivotably supported about a first rotational movement axis on the base frame;
wherein the robot arm further comprises a linkage that is pivotably supported relative to the carousel about a second rotational movement axis;
wherein the robot arm further comprises an arm section that relative to the linkage is pivotably supported about a third rotational movement axis that is horizontally arranged;
wherein the robot arm further comprises an intermediate section, wherein the intermediate sections secures the arm section on the linkage;
wherein the intermediate section is pivotably supported on the linkage about the third rotational movement axis; and
wherein the robot arm further comprises a hand section that enables movements of the patient support about the three spatial axes.

14. The patient positioning device according to claim 13, wherein the second rotational movement axis and the third rotational movement axis extend parallel to each other.

15. The patient positioning device according to claim 13, wherein the hand section is arranged on the arm section.

16. The patient positioning device according to claim 13, wherein the robot arm further comprises a coupling device, wherein the coupling device couples the movement of the intermediate section about the third rotational movement axis to the movement of the linkage about the second rotational movement axis.

17. The patient positioning device according to claim 16, wherein the coupling device comprises a coupling element, wherein the coupling element is arranged on the carousel so as to be pivotable about a first bearing axis and is arranged on the intermediate section so as to be pivotable about a second bearing axis, wherein the first bearing axis is positioned at a spacing relative to the second rotational movement axis and the second bearing axis is arranged at a spacing relative to the third rotational movement axis.

18. The patient positioning device according to claim 13, wherein the arm section is angled and comprises a first section secured on the intermediate section and further comprises a second section, wherein the hand section is arranged on the second section, wherein a spacing of a topside of the first section relative to the floor is greater than a spacing of the topside of the second section relative to the floor.

19. The patient positioning device according to claim 13, wherein the arm section relative to the intermediate section is pivotably supported about a fourth rotational movement axis that is vertically arranged.
